SEQUENCE LISTING (10 sequences)

SEQ ID NO: 1 - DNA, 2391 bp, synthetic construct
  misc_feature 1..2391: note = Synthetic Poilynucleotide
  source 1..2391: mol_type = other DNA
atgtcggcggcgcaggtgtcctcgtcccggagacaatcttgctacctgtgcgacctgccccgcatgccctgggccatgatctgggacttctcggaacccgtatgccgcggttgcgtcaactacgagggcgctgatcgcatcgaattcgtgatcgagacagcgcgccagctgaagcgggcgcacggctgcttccaggacggccgctcccccgggccgccgccgcccgtcggggtcaagacagtggccctgtcggctaaggaagcggcggcggcggcggcagcagcggcggccgccgccgccgccgcgcaacagcaacagcaacagcagcagcagcagcagcaacagcagcagcagcagcagcagcagcagcagcaacaacagctcaaccacgttgatggttccagcaagcctgcggtgctggcggccccgtctggcctggagcgctacggcctaagcgctgccgccgccgccgccgccgccgccgccgctgcggtggaacagcgcagccgcttcgagtacccgccaccgccggtgagcctgggaagcagcagccacaccgcgcgactgcccaacggcctggggggcccaaacggcttccccaaaccaacaccagaggagggacccccagagctgaaccgtcagagccccaattcttcttcagcggcggcgtcggtggcgtctcggcgtggaacgcacggtgggctggttacggggctgcccaacccggggggtggcggaggcccccagctcaccgtgccccccaacctgctaccgcagacgctgcttaacggcccggccagcgctgcggtactccccccaccccctccccacgccctgggcagccgtgggcccccgacgcctgctcccccaggggctcctgggggccccgcttgtctcgggggtaccccgggtgtatcggccacgtcgtcctccgcgtcgtcttcgacctcttcgtcggtggcagaggtgggcgtgggtgctggtggtaagaggcccggctcggtgtcgagcacagaccaggagcgcgagttgaaggagaagcagcgcaacgccgaggccctggccgagctgagcgagagcctgcgcaaccgcgccgaggagtgggccagcaagcccaagatggtccgcgacacgctgctcacgctggcaggctgcacgccctacgaggttcgcttcaagaaggaccactcgctgctgggccgcgttttcgccttcgacgccgtctccaagcccggcatggactacgaattgaagctgttcattgagtaccccacgggctcgggcaacgtgtactccagtgcatctggtgtggccaagcagatgtatcaggactgcatgaaggacttcggccggggcctatcctcgggtttcaagtacctggagtacgaaaagaagcacggctccggggactggcgcctgcttggagacctgctccccgaagccgtgcgcttcttcaaggagggcgtgcccggcgccgacatgctgccccagccctacctggacgccagctgtcccatgctgcccactgctctggtgagtctgagccgcgcccccagcgcacccccggggaccggggccttgccgcccgccgcgccgtcgggccggggcgcagccgccagcctgcgcaagagaaaggcctctccggagcccccggactcagccgagggcgcgctgaagctgggcgaggaacagcagaggcagcagtggatggcgaaccagagcgaggcgctgaagctcaccatgtccgccgggggcttcgcggcgccggggcacgcggcggggggtccgcctccgccgcccccacctctgggaccccattccaaccggaccaccccacctgagtcagccccccagaacggtccgtcccctatggccgctctcatgtcggtggcagatactctgggcacagcgcactcgcccaaggatggcagttccgtgcactctaccactgcgtcggcgcggcgaaacagcagcagcccagtctcgccggcctccgtgccggggcagcgccgcttggcatcacgtaacggggacctgaatttacaggtggcgcccccgccgcctagcgcccacccgggcatggaccaagtgcacccccaaaacattccggattcccccatggccaacagcggacccctctgctgcaccatttgccacgaacgtttggaggatacgcatttcgttcagtgcccttccgtccccagccacaaattttgcttcccttgctctagagagagtatcaaggcccagggggccaccggcgaggtgtattgccccagcggagagaaatgccccctagtcgggtcgaatgtaccttgggccttcatgcagggcgaaatcgcgactatcttagctggggatgttaaagtgaaaaaggagagagacccttga

SEQ ID NO: 2 - DNA, 4157 bp, synthetic construct
  misc_feature 1..4157: note = Synthetic Poilynucleotide
  source 1..4157: mol_type = other DNA
gcgagccgcagccggagcaggagcgccgagacgcgcctccggaacgtagagtaacaatcacaaccccacattccgggcgagcgtgagcacgagcgggaagggatgcgacccgggccgaggcgccgcgcgagcgccctgcagccaacgtgagcgccgccagccgcggcggcccgggcgccggccaggcctgggggcggcgggagcctgcgtgcgtgcactcctctcctctgctctcgtgcgtcctggaagcagtggccgcggcggctccctccggggtgcaaacccagtcgccgccagcagaacggccgacgctgcggaggggagaaggtcctttctcggctgccaccccctcccccggtcctccggggaagcagcggcttcagcaagattggacccgggcaccgggtggcactgaaccctctggccctcgccccagggggcccgtcggggagaggacgcagctcgtaggggggtccccggggagaggaagagacagcccctttcgagcttccacgcaccagccactccggggagggggccaagaggcaacggcggccaccaccgggcaccctcgccccctcccctcgggccgggagcttccagcccaagtctgcagcaccaggaagaaggcgcctgagctcccctcgcgacgagtcaaccgcagtaggaggtgggggcgaagagagggctgaacccgtccgctgcccgggcggtggagcccccacggcgaggcgctgcgccggcggtggagactcgcgttccctccagcccctggggcagaactttctcgccccccctcctccctcccccgcagtcggactccctccccagccggccagtcctcccggaggagaaggcgccgcggagacagcccgggcgggggcctaccttccccagggcaggcatcatgtcggcggcgcaggtgtcctcgtcccggagacaatcttgctacctgtgcgacctgccccgcatgccctgggccatgatctgggacttctcggaacccgtatgccgcggttgcgtcaactacgagggcgctgatcgcatcgaattcgtgatcgagacagcgcgccagctgaagcgggcgcacggctgcttccaggacggccgctcccccgggccgccgccgcccgtcggggtcaagacagtggccctgtcggctaaggaagcggcggcggcggcggcagcagcggcggccgccgccgccgccgcgcaacagcaacagcaacagcagcagcagcagcagcaacagcagcagcagcagcagcagcagcagcagcaacaacagctcaaccacgttgatggttccagcaagcctgcggtgctggcggccccgtctggcctggagcgctacggcctaagcgctgccgccgccgccgccgccgccgccgccgctgcggtggaacagcgcagccgcttcgagtacccgccaccgccggtgagcctgggaagcagcagccacaccgcgcgactgcccaacggcctggggggcccaaacggcttccccaaaccaacaccagaggagggacccccagagctgaaccgtcagagccccaattcttcttcagcggcggcgtcggtggcgtctcggcgtggaacgcacggtgggctggttacggggctgcccaacccggggggtggcggaggcccccagctcaccgtgccccccaacctgctaccgcagacgctgcttaacggcccggccagcgctgcggtactccccccaccccctccccacgccctgggcagccgtgggcccccgacgcctgctcccccaggggctcctgggggccccgcttgtctcgggggtaccccgggtgtatcggccacgtcgtcctccgcgtcgtcttcgacctcttcgtcggtggcagaggtgggcgtgggtgctggtggtaagaggcccggctcggtgtcgagcacagaccaggagcgcgagttgaaggagaagcagcgcaacgccgaggccctggccgagctgagcgagagcctgcgcaaccgcgccgaggagtgggccagcaagcccaagatggtccgcgacacgctgctcacgctggcaggctgcacgccctacgaggttcgcttcaagaaggaccactcgctgctgggccgcgttttcgccttcgacgccgtctccaagcccggcatggactacgaattgaagctgttcattgagtaccccacgggctcgggcaacgtgtactccagtgcatctggtgtggccaagcagatgtatcaggactgcatgaaggacttcggccggggcctatcctcgggtttcaagtacctggagtacgaaaagaagcacggctccggggactggcgcctgcttggagacctgctccccgaagccgtgcgcttcttcaaggagggcgtgcccggcgccgacatgctgccccagccctacctggacgccagctgtcccatgctgcccactgctctggtgagtctgagccgcgcccccagcgcacccccggggaccggggccttgccgcccgccgcgccgtcgggccggggcgcagccgccagcctgcgcaagagaaaggcctctccggagcccccggactcagccgagggcgcgctgaagctgggcgaggaacagcagaggcagcagtggatggcgaaccagagcgaggcgctgaagctcaccatgtccgccgggggcttcgcggcgccggggcacgcggcggggggtccgcctccgccgcccccacctctgggaccccattccaaccggaccaccccacctgagtcagccccccagaacggtccgtcccctatggccgctctcatgtcggtggcagatactctgggcacagcgcactcgcccaaggatggcagttccgtgcactctaccactgcgtcggcgcggcgaaacagcagcagcccagtctcgccggcctccgtgccggggcagcgccgcttggcatcacgtaacggggacctgaatttacaggtggcgcccccgccgcctagcgcccacccgggcatggaccaagtgcacccccaaaacattccggattcccccatggccaacagcggacccctctgctgcaccatttgccacgaacgtttggaggatacgcatttcgttcagtgcccttccgtccccagccacaaattttgcttcccttgctctagagagagtatcaaggcccagggggccaccggcgaggtgtattgccccagcggagagaaatgccccctagtcgggtcgaatgtaccttgggccttcatgcagggcgaaatcgcgactatcttagctggggatgttaaagtgaaaaaggagagagacccttgaaccactgggcagccacctcctttgccctagaccagctcctctccaatcctgagggcccctcccccaacccaactcgaccctccctcccctcacccccaaggtgtagaattgtgaatataacgaaactgcaaaaagttagtcttatgtatagacattattttcgtcgtatgtttctatattttgaaacaaaggtatgtaacttcttcatttgaaggataagctggtttgtgttaagcagtatagtattggttgggtcatttgcatcatatcgttagcatttatttggtggcagaatgtttgcctaggtacagaattaatagcccttagcaacgactgctgctggtgtgtatttttgtaaatgttatgcactctctgaaaggaaaaacacacacaaaagaaaaagacttttttttttttttttttttttttgccaaggccagtgttgctgcctaaaaaaaaaaaaaaaaaaaaaaaaaaaaaaaaaaaatgctataaaatggtgaaagcttccttctaaactgccccaagtgttgaagtcttcactttattttgttgttttgttttgtttttctgttttgtttgcaaaatggtaagggggtgtcgggggggatggggtgtattttgttgcaagtttgtgaggggaaaatgttttggtttgtttctactgacctgaatgtgttggatctacacgtgttgttttgtttttgctttattgatgcacggatgcttttgaacagtagagcgaaatgctagacatggagaatctgctctgtttgtcctttatacatttctgtagttaacagaacactgtaatgtgccttggagcttagtaacttgtaataaattcaattgatattaa

SEQ ID NO: 3 - DNA, 7740 bp, synthetic construct
  misc_feature 1..7740: note = Synthetic Poilynucleotide
  source 1..7740: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccaaaaatcgataaaaggcgcgccaaaaacgcgtgaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaacgcgtcgaagggcgaattctgcagataactggtaagtttagtcttttttgtcttttatttcaggtcccggatccggaaaggaaaggtcatcgtctgtttaatttccacccccagctctggacttccagcattagctcgccgggcctcagctgttgtacacacacggcgaggggggaggggagggcggaggcgcggaggaggggccggctgggaggaatctgggcgtcctgccgcgcatgcgccttgcctgagcaacaagtgggctccacagaggaagtgtaaaggggagggggaagaactggtgcagagcatggcggtgacgtcagcgctccgcccgggcggcatcccgcgcggccaagccggggacagcgcgagccgcagccggagcaggagcgccgagacgcgcctccggaacgtagagtaacaccggtgcgagccgcagccggagcaggagcgccgagacgcgcctccggaacgtagagtaacaatcacaaccccacattccgggcgagcgtgagcacgagcgggaagggatgcgacccgggccgaggcgccgcgcgagcgccctgcagccaacgtgagcgccgccagccgcggcggcccgggcgccggccaggcctgggggcggcgggagcctgcgtgcgtgcactcctctcctctgctctcgtgcgtcctggaagcagtggccgcggcggctccctccggggtgcaaacccagtcgccgccagcagaacggccgacgctgcggaggggagaaggtcctttctcggctgccaccccctcccccggtcctccggggaagcagcggcttcagcaagattggacccgggcaccgggtggcactgaaccctctggccctcgccccagggggcccgtcggggagaggacgcagctcgtaggggggtccccggggagaggaagagacagcccctttcgagcttccacgcaccagccactccggggagggggccaagaggcaacggcggccaccaccgggcaccctcgccccctcccctcgggccgggagcttccagcccaagtctgcagcaccaggaagaaggcgcctgagctcccctcgcgacgagtcaaccgcagtaggaggtgggggcgaagagagggctgaacccgtccgctgcccgggcggtggagcccccacggcgaggcgctgcgccggcggtggagactcgcgttccctccagcccctggggcagaactttctcgccccccctcctccctcccccgcagtcggactccctccccagccggccagtcctcccggaggagaaggcgccgcggagacagcccgggcgggggcctaccttccccagggcaggcatcatgtcggcggcgcaggtgtcctcgtcccggagacaatcttgctacctgtgcgacctgccccgcatgccctgggccatgatctgggacttctcggaacccgtatgccgcggttgcgtcaactacgagggcgctgatcgcatcgaattcgtgatcgagacagcgcgccagctgaagcgggcgcacggctgcttccaggacggccgctcccccgggccgccgccgcccgtcggggtcaagacagtggccctgtcggctaaggaagcggcggcggcggcggcagcagcggcggccgccgccgccgccgcgcaacagcaacagcaacagcagcagcagcagcagcaacagcagcagcagcagcagcagcagcagcagcaacaacagctcaaccacgttgatggttccagcaagcctgcggtgctggcggccccgtctggcctggagcgctacggcctaagcgctgccgccgccgccgccgccgccgccgccgctgcggtggaacagcgcagccgcttcgagtacccgccaccgccggtgagcctgggaagcagcagccacaccgcgcgactgcccaacggcctggggggcccaaacggcttccccaaaccaacaccagaggagggacccccagagctgaaccgtcagagccccaattcttcttcagcggcggcgtcggtggcgtctcggcgtggaacgcacggtgggctggttacggggctgcccaacccggggggtggcggaggcccccagctcaccgtgccccccaacctgctaccgcagacgctgcttaacggcccggccagcgctgcggtactccccccaccccctccccacgccctgggcagccgtgggcccccgacgcctgctcccccaggggctcctgggggccccgcttgtctcgggggtaccccgggtgtatcggccacgtcgtcctccgcgtcgtcttcgacctcttcgtcggtggcagaggtgggcgtgggtgctggtggtaagaggcccggctcggtgtcgagcacagaccaggagcgcgagttgaaggagaagcagcgcaacgccgaggccctggccgagctgagcgagagcctgcgcaaccgcgccgaggagtgggccagcaagcccaagatggtccgcgacacgctgctcacgctggcaggctgcacgccctacgaggttcgcttcaagaaggaccactcgctgctgggccgcgttttcgccttcgacgccgtctccaagcccggcatggactacgaattgaagctgttcattgagtaccccacgggctcgggcaacgtgtactccagtgcatctggtgtggccaagcagatgtatcaggactgcatgaaggacttcggccggggcctatcctcgggtttcaagtacctggagtacgaaaagaagcacggctccggggactggcgcctgcttggagacctgctccccgaagccgtgcgcttcttcaaggagggcgtgcccggcgccgacatgctgccccagccctacctggacgccagctgtcccatgctgcccactgctctggtgagtctgagccgcgcccccagcgcacccccggggaccggggccttgccgcccgccgcgccgtcgggccggggcgcagccgccagcctgcgcaagagaaaggcctctccggagcccccggactcagccgagggcgcgctgaagctgggcgaggaacagcagaggcagcagtggatggcgaaccagagcgaggcgctgaagctcaccatgtccgccgggggcttcgcggcgccggggcacgcggcggggggtccgcctccgccgcccccacctctgggaccccattccaaccggaccaccccacctgagtcagccccccagaacggtccgtcccctatggccgctctcatgtcggtggcagatactctgggcacagcgcactcgcccaaggatggcagttccgtgcactctaccactgcgtcggcgcggcgaaacagcagcagcccagtctcgccggcctccgtgccggggcagcgccgcttggcatcacgtaacggggacctgaatttacaggtggcgcccccgccgcctagcgcccacccgggcatggaccaagtgcacccccaaaacattccggattcccccatggccaacagcggacccctctgctgcaccatttgccacgaacgtttggaggatacgcatttcgttcagtgcccttccgtccccagccacaaattttgcttcccttgctctagagagagtatcaaggcccagggggccaccggcgaggtgtattgccccagcggagagaaatgccccctagtcgggtcgaatgtaccttgggccttcatgcagggcgaaatcgcgactatcttagctggggatgttaaagtgaaaaaggagagagacccttgaaagcttaataaaagatctttattttcattagatctgtgtgttggttttttgtgtgaaactcgagaagatatcaactgccttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcgttcttgactcttcgcgatgtacgggccagatatacgcgttgacattgattattgactagttattaatagtaatcaattacggggtcattagttcatagcccatatatggagttccgctcgagcaaaggcgcgccaaaaaagcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 4 - DNA, 7385 bp, synthetic construct
  misc_feature 1..7385: note = Synthetic Poilynucleotide
  source 1..7385: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccggttcctagatcagggcacaaaaacaaatagaaaagacaatttctggaagattggaaaacagctaattttgcaggtgcttcggcatcagtggtggcgatgaatttaacatccatgccgggggggataaggttcaaccgcagatgaaatggttaataccttatacgaaattcacggcgaaaaatcctgaaacaggacatttaaattcactgccgaggcttgggggtggggtggggagcagggatcggaaacgacgctagtggttcttaaatgcgtttttacttgggaatgcccggcagggaaaggaaaggtcatcgtctgtttaatttccacccccagctctggacttccagcattagctcgccgggcctcagctgttgtacacacacggcgaggggggaggggagggcggaggcgcggaggaggggccggctgggaggaatctgggcgtcctgccgcgcatgcgccttgcctgagcaacaagtgggctccacagaggaagtgtaaaggggagggggaagaactggtgcagagcatggcggtgacgtcagcgctccgcccgggcggcatcccgcgcggccaagccggggacagcgcgagccgcagccggagcaggagcgccgagacgcgcctccggaacgtagagtaacggatccgcgagccgcagccggagcaggagcgccgagacgcgcctccggaacgtagagtaacaatcacaaccccacattccgggcgagcgtgagcacgagcgggaagggatgcgacccgggccgaggcgccgcgcgagcgccctgcagccaacgtgagcgccgccagccgcggcggcccgggcgccggccaggcctgggggcggcgggagcctgcgtgcgtgcactcctctcctctgctctcgtgcgtcctggaagcagtggccgcggcggctccctccggggtgcaaacccagtcgccgccagcagaacggccgacgctgcggaggggagaaggtcctttctcggctgccaccccctcccccggtcctccggggaagcagcggcttcagcaagattggacccgggcaccgggtggcactgaaccctctggccctcgccccagggggcccgtcggggagaggacgcagctcgtaggggggtccccggggagaggaagagacagcccctttcgagcttccacgcaccagccactccggggagggggccaagaggcaacggcggccaccaccgggcaccctcgccccctcccctcgggccgggagcttccagcccaagtctgcagcaccaggaagaaggcgcctgagctcccctcgcgacgagtcaaccgcagtaggaggtgggggcgaagagagggctgaacccgtccgctgcccgggcggtggagcccccacggcgaggcgctgcgccggcggtggagactcgcgttccctccagcccctggggcagaactttctcgccccccctcctccctcccccgcagtcggactccctccccagccggccagtcctcccggaggagaaggcgccgcggagacagcccgggcgggggcctaccttccccagggcaggcatcatgtcggcggcgcaggtgtcctcgtcccggagacaatcttgctacctgtgcgacctgccccgcatgccctgggccatgatctgggacttctcggaacccgtatgccgcggttgcgtcaactacgagggcgctgatcgcatcgaattcgtgatcgagacagcgcgccagctgaagcgggcgcacggctgcttccaggacggccgctcccccgggccgccgccgcccgtcggggtcaagacagtggccctgtcggctaaggaagcggcggcggcggcggcagcagcggcggccgccgccgccgccgcgcaacagcaacagcaacagcagcagcagcagcagcaacagcagcagcagcagcagcagcagcagcagcaacaacagctcaaccacgttgatggttccagcaagcctgcggtgctggcggccccgtctggcctggagcgctacggcctaagcgctgccgccgccgccgccgccgccgccgccgctgcggtggaacagcgcagccgcttcgagtacccgccaccgccggtgagcctgggaagcagcagccacaccgcgcgactgcccaacggcctggggggcccaaacggcttccccaaaccaacaccagaggagggacccccagagctgaaccgtcagagccccaattcttcttcagcggcggcgtcggtggcgtctcggcgtggaacgcacggtgggctggttacggggctgcccaacccggggggtggcggaggcccccagctcaccgtgccccccaacctgctaccgcagacgctgcttaacggcccggccagcgctgcggtactccccccaccccctccccacgccctgggcagccgtgggcccccgacgcctgctcccccaggggctcctgggggccccgcttgtctcgggggtaccccgggtgtatcggccacgtcgtcctccgcgtcgtcttcgacctcttcgtcggtggcagaggtgggcgtgggtgctggtggtaagaggcccggctcggtgtcgagcacagaccaggagcgcgagttgaaggagaagcagcgcaacgccgaggccctggccgagctgagcgagagcctgcgcaaccgcgccgaggagtgggccagcaagcccaagatggtccgcgacacgctgctcacgctggcaggctgcacgccctacgaggttcgcttcaagaaggaccactcgctgctgggccgcgttttcgccttcgacgccgtctccaagcccggcatggactacgaattgaagctgttcattgagtaccccacgggctcgggcaacgtgtactccagtgcatctggtgtggccaagcagatgtatcaggactgcatgaaggacttcggccggggcctatcctcgggtttcaagtacctggagtacgaaaagaagcacggctccggggactggcgcctgcttggagacctgctccccgaagccgtgcgcttcttcaaggagggcgtgcccggcgccgacatgctgccccagccctacctggacgccagctgtcccatgctgcccactgctctggtgagtctgagccgcgcccccagcgcacccccggggaccggggccttgccgcccgccgcgccgtcgggccggggcgcagccgccagcctgcgcaagagaaaggcctctccggagcccccggactcagccgagggcgcgctgaagctgggcgaggaacagcagaggcagcagtggatggcgaaccagagcgaggcgctgaagctcaccatgtccgccgggggcttcgcggcgccggggcacgcggcggggggtccgcctccgccgcccccacctctgggaccccattccaaccggaccaccccacctgagtcagccccccagaacggtccgtcccctatggccgctctcatgtcggtggcagatactctgggcacagcgcactcgcccaaggatggcagttccgtgcactctaccactgcgtcggcgcggcgaaacagcagcagcccagtctcgccggcctccgtgccggggcagcgccgcttggcatcacgtaacggggacctgaatttacaggtggcgcccccgccgcctagcgcccacccgggcatggaccaagtgcacccccaaaacattccggattcccccatggccaacagcggacccctctgctgcaccatttgccacgaacgtttggaggatacgcatttcgttcagtgcccttccgtccccagccacaaattttgcttcccttgctctagagagagtatcaaggcccagggggccaccggcgaggtgtattgccccagcggagagaaatgccccctagtcgggtcgaatgtaccttgggccttcatgcagggcgaaatcgcgactatcttagctggggatgttaaagtgaaaaaggagagagacccttgaaagcttaataaaagatctttattttcattagatctgtgtgttggttttttgtgtgaaactcgagaagatatcaactgccttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcgttcttgactcttcgcgatgtacgggccagatatacgcgttgacattgattattgactagttattaatagtaatcaattacggggtcattagttcatagcccatatatggagttccgctcgagcaaaggcgcgccaaaaaagcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 5 - DNA, 546 bp, synthetic construct
  misc_feature 1..546: note = Synthetic Poilynucleotide
  misc_feature 1..546: note = P546 promoter
  source 1..546: mol_type = other DNA
gaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaa

SEQ ID NO: 6 - DNA, 270 bp, synthetic construct
  misc_feature 1..270: note = Synthetic Poilynucleotide
  misc_feature 1..270: note = hybrid chicken B -Actin promoter
  source 1..270: mol_type = other DNA
ccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggag

SEQ ID NO: 7 - DNA, 7170 bp, synthetic construct
  misc_feature 1..7170: note = Synthetic Polynucleotide
  source 1..7170: mol_type = other DNA
ggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcgcggccgcggtaccaaaaatcgataaaaggcgcgccaaaaacgcgtgaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaacgcgtcgaagggcgaattctgcagataactggtaagtttagtcttttttgtcttttatttcaggtcccggatccggtggtggtgcaaatcaaagaactgctcctcagtcgatgttgcctttacttctaggcctgtacggaaaccggtgcgagccgcagccggagcaggagcgccgagacgcgcctccggaacgtagagtaacaatcacaaccccacattccgggcgagcgtgagcacgagcgggaagggatgcgacccgggccgaggcgccgcgcgagcgccctgcagccaacgtgagcgccgccagccgcggcggcccgggcgccggccaggcctgggggcggcgggagcctgcgtgcgtgcactcctctcctctgctctcgtgcgtcctggaagcagtggccgcggcggctccctccggggtgcaaacccagtcgccgccagcagaacggccgacgctgcggaggggagaaggtcctttctcggctgccaccccctcccccggtcctccggggaagcagcggcttcagcaagattggacccgggcaccgggtggcactgaaccctctggccctcgccccagggggcccgtcggggagaggacgcagctcgtaggggggtccccggggagaggaagagacagcccctttcgagcttccacgcaccagccactccggggagggggccaagaggcaacggcggccaccaccgggcaccctcgccccctcccctcgggccgggagcttccagcccaagtctgcagcaccaggaagaaggcgcctgagctcccctcgcgacgagtcaaccgcagtaggaggtgggggcgaagagagggctgaacccgtccgctgcccgggcggtggagcccccacggcgaggcgctgcgccggcggtggagactcgcgttccctccagcccctggggcagaactttctcgccccccctcctccctcccccgcagtcggactccctccccagccggccagtcctcccggaggagaaggcgccgcggagacagcccgggcgggggcctaccttccccagggcaggcatcatgtcggcggcgcaggtgtcctcgtcccggagacaatcttgctacctgtgcgacctgccccgcatgccctgggccatgatctgggacttctcggaacccgtatgccgcggttgcgtcaactacgagggcgctgatcgcatcgaattcgtgatcgagacagcgcgccagctgaagcgggcgcacggctgcttccaggacggccgctcccccgggccgccgccgcccgtcggggtcaagacagtggccctgtcggctaaggaagcggcggcggcggcggcagcagcggcggccgccgccgccgccgcgcaacagcaacagcaacagcagcagcagcagcagcaacagcagcagcagcagcagcagcagcagcagcaacaacagctcaaccacgttgatggttccagcaagcctgcggtgctggcggccccgtctggcctggagcgctacggcctaagcgctgccgccgccgccgccgccgccgccgccgctgcggtggaacagcgcagccgcttcgagtacccgccaccgccggtgagcctgggaagcagcagccacaccgcgcgactgcccaacggcctggggggcccaaacggcttccccaaaccaacaccagaggagggacccccagagctgaaccgtcagagccccaattcttcttcagcggcggcgtcggtggcgtctcggcgtggaacgcacggtgggctggttacggggctgcccaacccggggggtggcggaggcccccagctcaccgtgccccccaacctgctaccgcagacgctgcttaacggcccggccagcgctgcggtactccccccaccccctccccacgccctgggcagccgtgggcccccgacgcctgctcccccaggggctcctgggggccccgcttgtctcgggggtaccccgggtgtatcggccacgtcgtcctccgcgtcgtcttcgacctcttcgtcggtggcagaggtgggcgtgggtgctggtggtaagaggcccggctcggtgtcgagcacagaccaggagcgcgagttgaaggagaagcagcgcaacgccgaggccctggccgagctgagcgagagcctgcgcaaccgcgccgaggagtgggccagcaagcccaagatggtccgcgacacgctgctcacgctggcaggctgcacgccctacgaggttcgcttcaagaaggaccactcgctgctgggccgcgttttcgccttcgacgccgtctccaagcccggcatggactacgaattgaagctgttcattgagtaccccacgggctcgggcaacgtgtactccagtgcatctggtgtggccaagcagatgtatcaggactgcatgaaggacttcggccggggcctatcctcgggtttcaagtacctggagtacgaaaagaagcacggctccggggactggcgcctgcttggagacctgctccccgaagccgtgcgcttcttcaaggagggcgtgcccggcgccgacatgctgccccagccctacctggacgccagctgtcccatgctgcccactgctctggtgagtctgagccgcgcccccagcgcacccccggggaccggggccttgccgcccgccgcgccgtcgggccggggcgcagccgccagcctgcgcaagagaaaggcctctccggagcccccggactcagccgagggcgcgctgaagctgggcgaggaacagcagaggcagcagtggatggcgaaccagagcgaggcgctgaagctcaccatgtccgccgggggcttcgcggcgccggggcacgcggcggggggtccgcctccgccgcccccacctctgggaccccattccaaccggaccaccccacctgagtcagccccccagaacggtccgtcccctatggccgctctcatgtcggtggcagatactctgggcacagcgcactcgcccaaggatggcagttccgtgcactctaccactgcgtcggcgcggcgaaacagcagcagcccagtctcgccggcctccgtgccggggcagcgccgcttggcatcacgtaacggggacctgaatttacaggtggcgcccccgccgcctagcgcccacccgggcatggaccaagtgcacccccaaaacattccggattcccccatggccaacagcggacccctctgctgcaccatttgccacgaacgtttggaggatacgcatttcgttcagtgcccttccgtccccagccacaaattttgcttcccttgctctagagagagtatcaaggcccagggggccaccggcgaggtgtattgccccagcggagagaaatgccccctagtcgggtcgaatgtaccttgggccttcatgcagggcgaaatcgcgactatcttagctggggatgttaaagtgaaaaaggagagagacccttgaaagcttaataaaagatctttattttcattagatctgtgtgttggttttttgtgtgaaactcgagaagatatcaactgccttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcgttcttgactcttcgcgatgtacgggccagatatacgcgttgacattgattattgactagttattaatagtaatcaattacggggtcattagttcatagcccatatatggagttccgctcgagcaaaggcgcgccaaaaaagcatgcggccgcgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaag

SEQ ID NO: 8 - DNA, 296 bp, synthetic construct
  misc_feature 1..296: note = Synthetic Polynucleotide
  source 1..296: mol_type = other DNA
ggaaaggaaaggtcatcgtctgtttaatttccacccccagctctggacttccagcattagctcgccgggcctcagctgttgtacacacacggcgaggggggaggggagggcggaggcgcggaggaggggccggctgggaggaatctgggcgtcctgccgcgcatgcgccttgcctgagcaacaagtgggctccacagaggaagtgtaaaggggagggggaagaactggtgcagagcatggcggtgacgtcagcgctccgcccgggcggcatcccgcgcggccaagccggggacagc

SEQ ID NO: 9 - DNA, 648 bp, synthetic construct
  misc_feature 1..648: note = Synthetic Polynucleotide
  source 1..648: mol_type = other DNA
gctggggggaagggtagcgcactctgaattcatttagctctatcgttgaagcccgggttcctagatcagggcacaaaaacaaatagaaaagacaatttctggaagattggaaaacagctaattttgcaggtgcttcggcatcagtggtggcgatgaatttaacatccatgccgggggggataaggttcaaccgcagatgaaatggttaataccttatacgaaattcacggcgaaaaatcctgaaacaggacatttaaattcactgccgaggcttgggggtggggtggggagcagggatcggaaacgacgctagtggttcttaaatgcgtttttacttgggaatgcccggcagggaaaggaaaggtcatcgtctgtttaatttccacccccagctctggacttccagcattagctcgccgggcctcagctgttgtacacacacggcgaggggggaggggagggcggaggcgcggaggaggggccggctgggaggaatctgggcgtcctgccgcgcatgcgccttgcctgagcaacaagtgggctccacagaggaagtgtaaaggggagggggaagaactggtgcagagcatggcggtgacgtcagcgctccgcccgggcggcatcccgcgcggccaagccggggacagc

SEQ ID NO: 10 - DNA, 1034 bp, synthetic construct
  misc_feature 1..1034: note = Synthetic Polynucleotide
  source 1..1034: mol_type = other DNA
cgaggcagaggttgcagtgagccgagatcgcgccattttgcactccagcctgggcgacagagcaagactccgtcttaaaaaaaaaaaaaaaaaaaaatcacggccttgttccgccttctggggcaacatggagtgagatcacagcgcctgagaaagaactttgaaaactgcggctgtgctttacaaaaatgtatcagtattattaaatgctgcctccaccttttaaaaatcaaactctagaaaaaacactcacctgtttggcctctccggcccctccccctccctgccctgaactgcagctgtgcgcagccagttgcctgttttccttaaaggcctcggggtgctcgtatcttcttcagacgttcagccaaaatttttaaagtgcggctggggggaagggtagcgcactctgaattcatttagctctatcgttgaagcccgggttcctagatcagggcacaaaaacaaatagaaaagacaatttctggaagattggaaaacagctaattttgcaggtgcttcggcatcagtggtggcgatgaatttaacatccatgccgggggggataaggttcaaccgcagatgaaatggttaataccttatacgaaattcacggcgaaaaatcctgaaacaggacatttaaattcactgccgaggcttgggggtggggtggggagcagggatcggaaacgacgctagtggttcttaaatgcgtttttacttgggaatgcccggcagggaaaggaaaggtcatcgtctgtttaatttccacccccagctctggacttccagcattagctcgccgggcctcagctgttgtacacacacggcgaggggggaggggagggcggaggcgcggaggaggggccggctgggaggaatctgggcgtcctgccgcgcatgcgccttgcctgagcaacaagtgggctccacagaggaagtgtaaaggggagggggaagaactggtgcagagcatggcggtgacgtcagcgctccgcccgggcggcatcccgcgcggccaagccggggacagc

(19) United States
(12) Patent Application Publication
(10) Pub. No.: US 2026/0193675 A1
(43) Pub. Date: Jul. 9, 2026

(54) MATERIALS AND METHODS FOR THE TREATMENT OF DISORDERS ASSOCIATED WITH THE IRF2BPL GENE

(71) Applicant: RESEARCH INSTITUTE AT NATIONWIDE CHILDREN'S HOSPITAL, Columbus, OH (US)

(72) Inventors: Nicolas Sebastien Wein, Columbus, OH (US); Kathrin Christine Meyer, Columbus, OH (US)

(21) Appl. No.: 19/575,485

(22) Filed: Mar. 23, 2026

Related U.S. Application Data
(63) Continuation of application No. 17/769,933, filed on Oct. 19, 2020, now Pat. No. 12,606,846.
(60) Provisional application No. 62/923,432, filed on Oct. 18, 2019.

(51) Int. Cl. C12N 15/86; C12N 15/113

(52) U.S. Cl. CPC C12N 15/86; C12N 15/113; C12N 2750/14143

(57) ABSTRACT

The disclosure provides gene therapy vectors, such as adeno-associated virus (AAV), designed for treatment of an Interferon regulatory factor 2 binding protein like (IRF2BPL) disorder.

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: 54846A_SeqListing.xml; Size: 46,029 bytes; Created: Mar. 16, 2026.

### FIELD OF THE INVENTION

The disclosure provides gene therapy vectors, such as adeno-associated virus (AAV), designed for treatment of disorders caused by mutation in the Interferon regulatory factor 2 binding protein like (IRF2BPL). The disclosed AAV provide a healthy copy of the IRF2BPL cDNA able to produce the functional protein.

### BACKGROUND

The Interferon Regulatory Factor 2 Binding Protein Like (IRF2BPL) gene encodes a member of the IRF2BP family of transcriptional regulators. The deduced 796-amino acid protein has a calculated molecular mass of 82.7 kD. It is proline-rich and contains N-terminal polyglutamine and polyalanine tracts, a C-terminal C3HC4-type ring finger domain, and 2 putative transmembrane domains. It also has a potential nuclear targeting signal, an endoplasmic reticulum retention signal, 3 possible PEST sequences, and putative sites for phosphorylation, N-glycosylation, and amidation. RT-PCR analysis of human tissues detected strong expression in heart, moderate expression in skeletal muscle and pancreas, and weak expression in brain, kidney, liver, testis, thyroid, and lymphocytes. There are two mammalian paralogs IRF2BP1 and IRF2BP2 that share two highly conserved domains. Using DNA microarrays, Heger et al. (2007) found that expression of IRF2BPL, which they termed Eap1, increased in the medial basal hypothalamus, but not cerebral cortex, of female rhesus monkeys at early puberty and increased further at mid-puberty. Female mice underwent a similar increase of Eap1 expression in hypothalamus, but not cortex, during puberty. The function of this protein in unknown, and until recently it was not associated to a Mendelian disease.

Mutations in the IRF2BPL gene were found to be associated with NEDAMSS (neurodevelopmental disorder with regression, abnormal movements, loss of speech, and seizures) disease in children. NEDAMSS exhibits autosomal dominant inheritance that has onset during childhood and is a progressive disorder. There are no current therapies for NEDAMSS and any other IRF2BPL disorders and there is a need to develop such therapies.

### SUMMARY

In one aspect, described herein is a polynucleotide comprising (a) one or more regulatory control elements; and (b) an Interferon regulatory factor 2 binding protein like (IRF2BPL) cDNA sequence. In some embodiments, the regulatory control element is pIRF promoter, p546 promoter comprising a nucleotide sequence set forth in SEQ ID NO: 5 or CBA promoter comprising a nucleotide sequence set forth in SEQ ID NO: 6, or fragments thereof which retain regulatory control or promoter activity. In some embodiments, the IRF2BPL cDNA comprises the polynucleotide sequence set forth in SEQ ID NO: 1.

In another aspect, described herein is a recombinant adeno-associated virus (rAAV) having a genome comprising a polynucleotide sequence described herein. In some embodiments, the rAAV is of the serotype AAV1, AAV2, AAV3, AAV4, AAV5, AAV6, AAV7, AAV8, AAV9, AAVRH10, AAVRH74, AAV11, AAV12, AAV13, or Anc80, AAV7m8 and their derivatives.

In some embodiments, the genome of the rAAV comprises a pIRF promoter fragment and an IRF2BPL cDNA. The nucleotide sequence of the pIRF promoter fragment is 1034 base pairs and set out as SEQ ID NO: 10. For example, the genome of the rAAV comprises a pIRF promoter comprising nucleotides 1-1034 of the pIRF (SEQ ID NO: 10), or a promoter fragment comprising nucleotides 386-1034 of the pIRF (SEQ ID NO: 9), or a promoter fragment comprising nucleotides 738-1034 of the pIRF (SEQ ID NO: 8).

In some embodiments, the genome of the rAAV comprises a p546 promoter and an IRF2BPL cDNA.

In some embodiments, the genome of the rAAV comprises a CBA promoter and an IRF2BPL cDNA.

In some embodiments, the genome of the rAAV comprises a pIRF promoter fragment, a p546 promoter and an IRF2BPL cDNA, wherein the promoter retains promoter activity.

In an exemplary embodiment, the genome of the rAAV comprises nucleotides 342 to 5299 of SEQ ID NO: 3, or nucleotides 342 to 5031 of SEQ ID NO: 4, or nucleotides 342 to 5070 of SEQ ID NO: 7.

In another aspect, described herein is an rAAV particle comprising an rAAV described herein.

Methods of treating an Interferon regulatory factor 2 binding protein like (IRF2BPL)-related disorder in a subject in need thereof comprising administering an rAAV or an rAAV particle described herein are specifically contemplated. An IRF2BPL-related disorder is a neurological disorder that is associated with the presence of a mutation in the IRF2BPL gene. In some embodiments, the IRF2BPL-related disorder comprises NEDAMSS (neurodevelopmental disorder with regression, abnormal movements, loss of speech, and seizures) or other neurological disorders including, but not limited to, epilepsy, schizophrenia and neuropathy.

In another aspect, described herein is the use an rAAV or an rAAV particle described herein in the preparation of a medicament for the treatment of an Interferon regulatory factor 2 binding protein like (IRF2BPL)-related disorder, such as NEDAMSS.

In another aspect, described herein is a composition comprising an rAAV or an rAAV particle described herein for the treatment of an Interferon regulatory factor 2 binding protein like (IRF2BPL)-related disorder, such as NEDAMSS.

### BRIEF DESCRIPTION OF THE FIGURES

FIG. 1 is a schematic of the IRF2BPL protein domains and the location of the mutations that are present in the patient derived skin fibroblasts used in this study.

FIG. 2A-2B provides data demonstrating the expression of Interferon regulatory factor 2 binding protein like (IRF2BPL) in fibroblasts isolated from healthy individuals and patients suffering from NEDAMSS (denoted as “disease”). FIG. 2A demonstrates IRF2BPL protein expression detected by immunofluorescence. FIG. 2B provides IRF2BPL protein expression detected by Western blot (n=3). The data indicates no major differences in protein expression levels between healthy controls and NEDAMSS patients except for one patient (P3) which shows a reduction.

FIG. 3 provides graphs showing the expression of IRF2BPL protein in astrocytes (iAST) induced from fibroblasts isolated from healthy individuals and patients suffering from NEDAMSS. No major differences were seen in overall levels of protein expression except for the same patient cell line in which already the fibroblasts showed a decrease in IRF2BPL expression in FIG. 2 (P3).

FIGS. 4A and 4B are representative images showing that IRF2BPL aberrantly accumulates in the cytoplasm of iAST from patients suffering from NEDAMSS (P1. P2, P3 and P4). The blue stain is DAPI and the red staining is IRF2BPL, when overlapping, the stain appears purple.

FIG. 5 provides a graph showing the normalized ratio of number of cells with cytoplasm accumulation of IRF2BPL in fibroblasts to the DAPI counts (n=3).

FIG. 6 provides graphs showing IRF2BPL protein expression in nucleus (FIG. 6A) and cytoplasm (FIG. 6B) extracts from patients and control astrocyte (n=3). Also supporting the increase in accumulation in the cytoplasm in patient iASTs and a decrease in nuclear localization.

FIG. 7A-7B provides representative images and quantification showing mouse motor neuron survival after three days co-culture of patient or healthy iASTs with GFP+ motor neurons (in black), n=4.

FIG. 8 provides immunofluorescence staining for WNT1 and DAPI in NEDAMSS patient cell lines. WNT1 expression was increased in the NEDAMSS patients.

FIG. 9 provides Western blot data measuring WNT1 levels in the lysate and the supernatant in ASTs from healthy and NEDAMSS patients (n=1). The graph demonstrates that the WNT1 expression is increased in the astrocyte supernatants from NEDAMSS patients.

FIGS. 10A and B provide representative images showing that reduced numbers of neurons were found in NEDAMSS patient cell lines compared to healthy controls after direct reprogramming from fibroblasts. FIG. 10A provides staining using the pan-neuronal marker Tuj1, and FIG. 10B provides staining for neuron specific marker GABA. Control refers to neurons induced from a healthy individual.

FIG. 11 provides graphs showing the comparison of percentage neuronal conversion rate and the neurite length. Indicating reduced generation of neurons from fibroblasts of NEDAMSS patients.

FIGS. 12A and 13B are graphs showing IRF2BPL mRNA (FIG. 12A) and IRF2BPL protein (FIG. 12B) expression between wild-type IRF2BPL levels in HEK-293 and GFP levels as induced by five different promoters.

FIG. 13 depicts the strategy for designing three AAV gene therapy vectors encoding the wild type IRE2BPL gene using p546 and truncated variants of the endogenous pIRF promoter.

FIG. 14 provides an annotated sequence of ssAAV9-p546+pIRFENH_5′UTR-IRF2BPL (SEQ ID NO: 3) showing the location of the construct elements.

FIG. 15 provides an annotated sequence of ssAAV9-pIRFSHORT_5′UTR-IRF2BPL (SEQ ID NO: 4) showing the location of the construct elements.

FIG. 16 provides an annotated sequence of ssAAV9-p546-5′UTR-IRF2BPL SEQ ID NO: 7) showing the location of the construct elements.

### DETAILED DESCRIPTION

The Interferon Regulatory Factor 2 Binding Protein Like (IRF2BPL) gene encodes a member of the IRF2BP family of transcriptional regulators.

IRF2BPL is an intron-less gene located at 14q24.23. The transcript is expressed in many organs, including in the central nervous system (CNS) components such as the cerebellum.

### IRF2BPL Mutations

The wild-type cDNA sequence of IRF2BPL is set forth in SEQ ID NO: 1. The nucleic acid sequence of the full length IRF2BPL gene is set for the in SEQ ID NO: 2. The in vivo function of IRF2BPL in all species remains largely undefined although previous studies have shown its role in the reproductive cycle in rodents and monkeys. Recently, mutations in this gene were found to be associated with NEDAMSS (neurodevelopmental disorder with regression, abnormal movements, loss of speech, and seizures) disease in children. NEDAMSS exhibits autosomal dominant inheritance. Not much is known regarding the mechanism of this rare disease. A total of 18 patients are known to have this disease so far in the world.

In some embodiments, the IRF2BPL gene in a cell of a diseased subject comprises one or of the following mutations in SEQ ID NO: 1: Q126X, Q127X, E172X, Y173X, R188X, G195V, P372R, K418N, and/or a frame shift mutation at A708 (A708fs).

### AAV Gene Therapy

The present disclosure provides for gene therapy vectors, e.g. rAAV vectors, expressing the IRF2BPL gene and methods of treating NEDAMSS (neurodevelopmental disorder with regression, abnormal movements, loss of speech, and seizures).

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle.

Adeno-associated virus (AAV) is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45:555-564 (1983) as corrected by Ruffing et al., J Gen Virol, 75:3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78:6381-6388 (2004); the AAV-10 genome is provided in Mol. Ther., 13 (1): 67-76 (2006); and the AAV-11 genome is provided in Virology, 330 (2): 375-383 (2004). Cloning of the AAVrh.74 serotype is described in Rodino-Klapac, et al. Journal of translational medicine 5, 45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° C. to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8:659-669 (1997); Kessler et al., Proc Nat. Acad Sc. USA, 93:14082-14087 (1996); and Xiao et al., J Virol, 70:8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94:5804-5809 (1997) and Murphy et al., Proc Natl Acad Sci USA, 94:13921-13926 (1997). Moreover, Lewis et al., J Virol, 76:8769-8775 (2002) demonstrated that skeletal myofibers possess the necessary cellular factors for correct antibody glycosylation, folding, and secretion, indicating that muscle is capable of stable expression of secreted protein therapeutics.

Recombinant AAV genomes of the disclosure comprise nucleic acid molecule of the disclosure and one or more AAV ITRs flanking a nucleic acid molecule. AAV DNA in the rAAV genomes may be from any AAV serotype for which a recombinant virus can be derived including, but not limited to, AAV serotypes (e.g., AAV1, AAV2, AAV3, AAV4, AAV5, AAV6, AAV7, AAV8, AAV9, AAVRH10, AAVRH74, AAV11, AAV12, AAV13, or Anc80, AAV7m8 and their derivatives). Production of pseudotyped rAAV is disclosed in, for example, WO 01/83692. Other types of rAAV variants, for example rAAV with capsid mutations, are also contemplated. See, for example, Marsic et al., Molecular Therapy, 22 (11): 1900-1909 (2014). As noted in the Background section above, the nucleotide sequences of the genomes of various AAV serotypes are known in the art.

The provided recombinant AAV (i.e., infectious encapsidated rAAV particles) comprise a rAAV genome. The term “rAAV genome” refers to a polynucleotide sequence that is derived from a native AAV genome that has been modified. In some embodiments, the rAAV genome has been modified to remove the native cap and rep genes. In some embodiments, the rAAV genome comprises the endogenous 5′ and 3′ inverted terminal repeats (ITRs). In some embodiments, the rAAV genome comprises ITRs from an AAV serotype that is different from the AAV serotype from which the AAV genome was derived. In some embodiments, the rAAV genome comprises a transgene of interest flanked on the 5′ and 3′ ends by inverted terminal repeat (ITR). In some embodiments, the rAAV genome comprises a “gene cassette.” In exemplary embodiments, the genomes of both rAAV lack AAV rep and cap DNA, that is, there is no AAV rep or cap DNA between the ITRs of the genomes.

The rAAV genomes provided herein, in some embodiments, comprise one or more AAV ITRs flanking the transgene polynucleotide sequence. The transgene polynucleotide sequence is operatively linked to transcriptional control elements (including, but not limited to, promoters, enhancers and/or polyadenylation signal sequences) that are functional in target cells to form a gene cassette. Examples of promoters are the pIRF promoter (SEQ ID NO: 10), chicken β actin promoter (CBA) comprising the polynucleotide sequence set forth in SEQ ID NO: 6, and the P546 promoter comprising the polynucleotide sequence set forth in SEQ ID NO: 5. Additional promoters are contemplated herein including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-la promoter, the hemoglobin promoter, and the creatine kinase promoter.

Additionally provided herein are a pIRF promoter sequence, a CB promoter sequence, a P546 promoter sequence, and promoter sequences at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence of the pIRF (SEQ ID NO: 10), CBA (SEQ ID NO: 6) or P546 (SEQ ID NO: 5) sequence which exhibit transcription promoting activity.

Other examples of transcription control elements are tissue specific control elements, for example, promoters that allow expression specifically within neurons or specifically within astrocytes. Examples include neuron specific enolase and glial fibrillary acidic protein promoters. Inducible promoters are also contemplated. Non-limiting examples of inducible promoters include, but are not limited to a metallothionine promoter, a glucocorticoid promoter, a progesterone promoter, and a tetracycline-regulated promoter. The gene cassette may also include intron sequences to facilitate processing of a transgene RNA transcript when expressed in mammalian cells. One example of such an intron is the SV40 intron.

rAAV genomes provided herein comprises a polynucleotide (SEQ ID NO: 1) encoding IRF2BPL. In some embodiments, the rAAV genomes provided herein comprises a polynucleotide sequence that encodes a polypeptide comprising an amino acid sequence that is at least: 90%, 91%, 92%, 93%, 94%, 95%, 96%, 97%, 98%, or 99% identical to the amino acid sequence encoded by the IRF2BPL cDNA (SEQ ID NO 1).

rAAV genomes provided herein, in some embodiments, a polynucleotide sequence that encodes an IRF2BPL protein and that hybridizes under stringent conditions to the polynucleotide sequence set forth in SEQ ID NO: 1 or the complement thereof.

In exemplary embodiments, the rAAV genomes comprises nucleotides 342 to 5299 of SEQ ID NO: 3, or nucleotides 342 to 5031 of SEQ ID NO: 4, or nucleotides 342 to 5070 of SEQ ID NO: 7. In some embodiments, the rAAV genomes provided herein comprise a polynucleotide sequences that is at least: 90%, 91%, 92%, 93%, 94%, 95%, 96%, 97%, 98%, or 99% identical to nucleotides 342 to 5299 of SEQ ID NO: 3, or nucleotides 342 to 5031 of SEQ ID NO: 4, or nucleotides 342 to 5070 of SEQ ID NO: 7. In other embodiments, the rAAV genomes provided herein comprise a polynucleotide sequence that hybridizes under stringent conditions to nucleotides 342 to 5299 of SEQ ID NO: 3, or nucleotides 342 to 5031 of SEQ ID NO: 4, or nucleotides 342 to 5070 of SEQ ID NO: 7.

DNA plasmids of the disclosure comprise rAAV genomes of the disclosure. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-9, AAV-1, AAV-2, AAV-3, AAV-4, AAV-5, AAV-6, AAV-7, AAVrh.74, AAV-8, AAV-10, AAV-11, AAV-12 and AAV-13. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial, and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13:1244-1250 (1995); Paul et al. Human Gene Therapy 4:609-615 (1993); Clark et al. Gene Therapy 3:1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV production.

The disclosure thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10 (6): 1031-1039 (1999); Schenpp and Clark, Methods Mol. Med., 69 427-443 (2002); U.S. Pat. No. 6,566,118 and WO 98/09657.

Compositions provided herein comprise rAAV and a pharmaceutically acceptable excipient or excipients. Acceptable excipients are nontoxic to recipients and are preferably inert at the dosages and concentrations employed, and include, but are not limited to, buffers such as phosphate [e.g., phosphate-buffered saline (PBS)], citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, copolymers such as poloxamer 188, pluronics (e.g., Pluronic F68) or polyethylene glycol (PEG). Compositions provided herein can comprise a pharmaceutically acceptable aqueous excipient containing a non-ionic, low-osmolar compound such as iobitridol, iohexol, iomeprol, iopamidol, iopentol, iopromide, ioversol, or ioxilan, where the aqueous excipient containing the non-ionic, low-osmolar compound can have one or more of the following characteristics: about 180 mgI/mL, an osmolality by vapor-pressure osmometry of about 322 mOsm/kg water, an osmolarity of about 273 mOsm/L, an absolute viscosity of about 2.3 cp at 20° C. and about 1.5 cp at 37° C., and a specific gravity of about 1.164 at 37° C. Exemplary compositions comprise about 20 to 40% non-ionic, low-osmolar compound or contrast agent or about 25% to about 35% non-ionic, low-osmolar compound. An exemplary composition comprises scAAV or rAAV viral particles formulated in 20 mM Tris (pH8.0), 1 mM MgCl2, 200 mM NaCl, 0.001% poloxamer 188 and about 25% to about 35% non-ionic, low-osmolar compound. Another exemplary composition comprises scAAV formulated in and 1×PBS and 0.001% Pluronic F68.

Dosages of rAAV to be administered in methods of the disclosure will vary depending, for example, on the particular rAAV, the mode of administration, the time of administration, the treatment goal, the individual, and the cell type(s) being targeted, and may be determined by methods standard in the art. Dosages may be expressed in units of viral genomes (vg). Dosages contemplated herein include about 1×10⁷, 1×10⁸, 1×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 1×10¹¹, about 1×10¹², about 1×10¹³, about 1.1×10¹³, about 1.2×10¹³, about 1.3×10¹³, about 1.5×10¹³, about 2×10¹³, about 2.5×10¹³, about 3×10¹³, about 3.5×10¹³, about 4×10¹³, about 4.5×10¹³, about 5×10¹³, about 6×10¹³, about 1×10¹⁴, about 2×10¹⁴, about 3×10¹⁴, about 4×10¹⁴ about 5×10¹⁴, about 1×10¹⁵, to about 1×10¹⁶, or more total viral genomes. Dosages of about 1×10⁹ to about 1×10¹⁰, about 5×10⁹ to about 5×10¹⁰, about 1×10¹⁰ to about 1×10¹¹, about 1×10¹¹ to about 1×10¹⁵ vg, about 1×10¹² to about 1×10¹⁵ vg, about 1×10¹² to about 1×10¹⁴ vg, about 1×10¹³ to about 6×10¹⁴ vg, and about 6×10¹³ to about 1.0×10¹⁴ vg are also contemplated. One dose exemplified herein is 6×10¹³ vg. Another dose exemplified herein is 1.5×10¹³ vg.

Methods of transducing a target cell with rAAV, in vivo or in vitro, are contemplated by the disclosure. The in vivo methods comprise the step of administering an effective dose, or effective multiple doses, of a composition comprising a rAAV of the disclosure to an animal (including a human being) in need thereof. If the dose is administered prior to development of a disorder/disease, the administration is prophylactic. If the dose is administered after the development of a disorder/disease, the administration is therapeutic. In embodiments of the disclosure, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with methods of the disclosure is NEDAMSS.

Combination therapies are also contemplated by the disclosure. Combination as used herein includes both simultaneous treatment and sequential treatments. Combinations of methods of the disclosure with standard medical treatments are specifically contemplated, as are combinations with novel therapies.

Administration of an effective dose of the compositions may be by routes standard in the art including, but not limited to, intramuscular, parenteral, intrathecal or other methods of accessing the cerborspinal fluid, inracerebroventricular, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the disclosure may be chosen and/or matched by those skilled in the art taking into account the infection and/or disease state being treated and the target cells/tissue(s) that are to express the wild type IRF2BPL protein.

The disclosure provides for local administration and systemic administration of an effective dose of rAAV and compositions of the disclosure. For example, systemic administration is administration into the circulatory system so that the entire body is affected. Systemic administration includes enteral administration such as absorption through the gastrointestinal tract and parenteral administration through injection, infusion or implantation.

Transduction of cells with rAAV of the disclosure results in sustained expression of the IRF2BPL protein. The present disclosure thus provides methods of administering/delivering rAAV which express IRF2BPL protein to an animal, preferably a human being. These methods include transducing cells with one or more rAAV of the present disclosure.

The term “transduction” is used to refer to the administration/delivery of the coding region of the IRF2BPL to a recipient cell either in vivo or in vitro, via a replication-deficient rAAV of the disclosure resulting in expression of IRF2BPLby the recipient cell.

The following EXAMPLES are provided by way of illustration and not limitation. Described numerical ranges are inclusive of each integer value within each range and inclusive of the lowest and highest stated integer.

### EXAMPLES

### Example 1—Characterization of iAstrocytes from NEDAMSS Patients

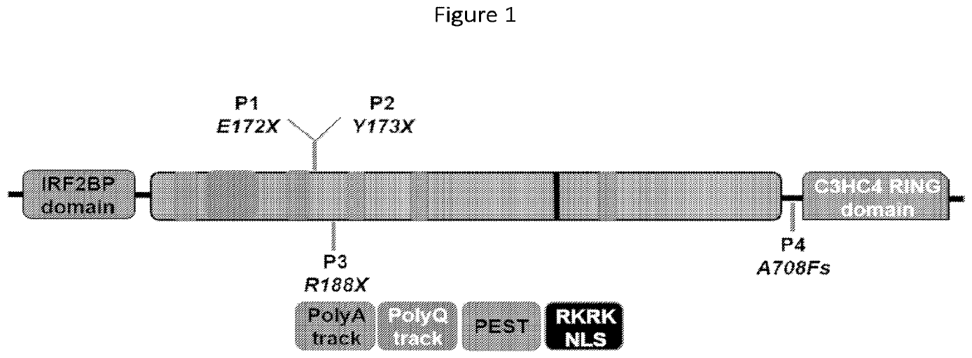

Fibroblasts from patients suffering from NEDAMSS (neurodevelopmental disorder with regression, abnormal movements, loss of speech, and seizures) were converted to induced neuronal progenitor cells (iNPCs) as previously described (Meyer et al., PNAS 829-832 (2014)). The fibroblasts were obtained from 4 families having nonsense variants in the IRF2BPL gene resulting in the truncation of its RING finger domain. The IRF2BPL gene mutations represented are summarized below and FIG. 1:

| Family | Mutation | Unaffected family members |
|---|---|---|
| 1 | Proband with E172X | Unaffected parents |
| | and G195V | |
| 2 | Proband with Y173X | Three unaffected family |
| | variant | members (parents + sib) |
| 3 | Proband with R188X | Unaffected parents |
| | variant | |
| 4 | Proband with A708fs | Three unaffected family |
| | variant | members (parent + sib) |

| Cell Line | Description |
|---|---|
| H1 | Healthy unrelated boy (S3) |
| H2 | Healthy sister (542) |
| H3 | Healthy adult female (AG) |
| H4 | Healthy adult male (fTM154) |
| P1 | Child patient with stop codon mutation near |
| | N terminal (537) |
| P2 | Child patient with stop codon mutation near |
| | N terminal (152) |
| P3 | Adult patient with stop codon mutation near |
| | N terminal (1911) |
| P4 | Child patient with frameshift mutation near |
| | C terminal (645) |

Neuronal progenitors' cells were cultured on fibronectin coated dishes in NPC media (DMEM/F12 media containing 1% N2 supplement (Life Technologies), 1% B27, 1% Anti-anti (antibiotic-antimycotic) 20 ng/ml fibroblast growth factor-2) until confluent. iAstrocytes were differentiated by seeding a small quantity of NPCs on another fibronectin coated dish in astrocyte inducing media (DMEM media containing 0.2% N2). These induced astrocytes are referred to as iastrocytes or iAST herein. Five days post differentiation, induced astrocytes were seeded either into a 96 well (10,000 cells/well), 384 well (2,500 cells/well), a 24 well seahorse plate (20,000 cells/well) or a 96 well seahorse plate (10,000 cells/well).

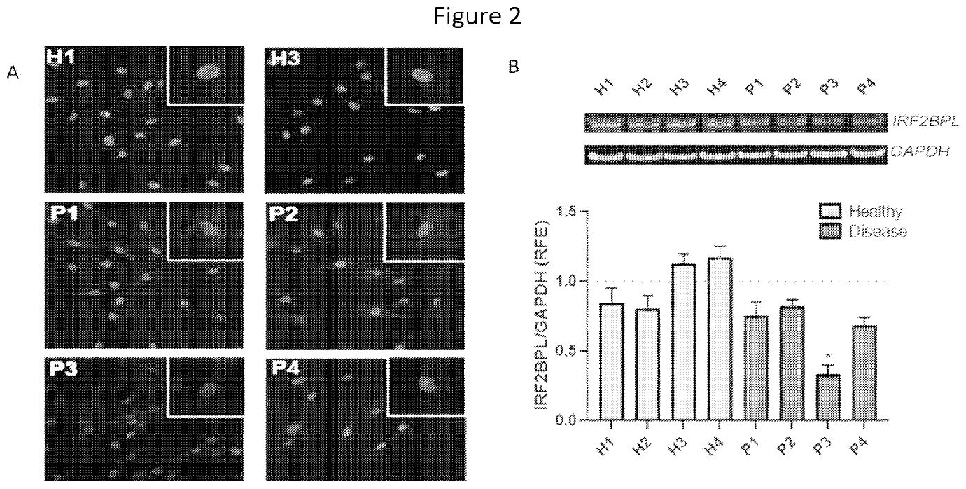

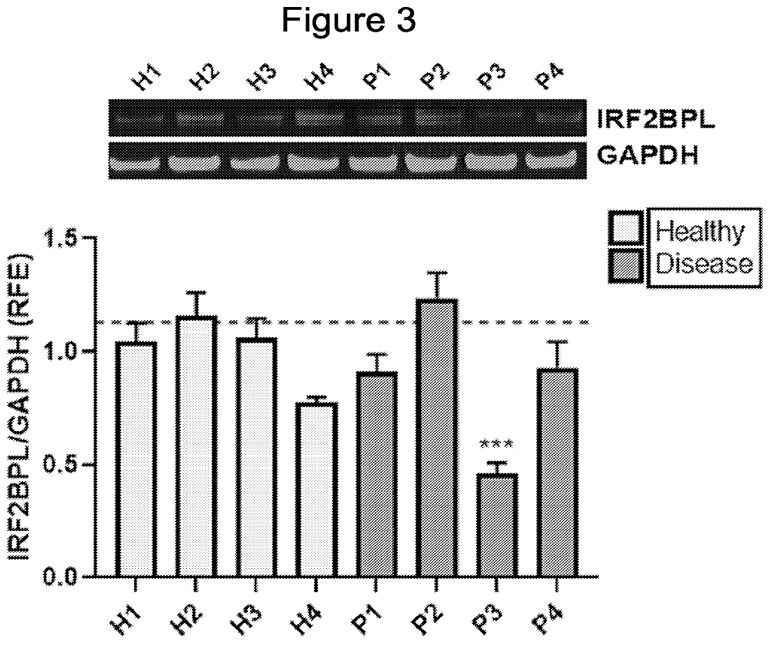

Immunohistochemistry was carried out on the primary fibroblasts from NEDAMSS patients and the iastrocytes from the same patients using an antibody specific for IRF2BPL (Novus Biologics). As shown in FIG. 2, expression of the IRF2BPL protein was not significantly different in the primary fibroblasts from the NEDAMSS patients (P1, P2 and P4) except the cells from patient P3 showed reduced expression, compared to fibroblasts from healthy individuals (H1, H2, H3 and H4). Similarly, as shown in FIG. 3, expression levels of the IRF2BPL protein was similar in the iastrocytes from the NEDAMSS patients and the heathy individuals, except for the cell line from patient P3 which showed significantly lower expression.

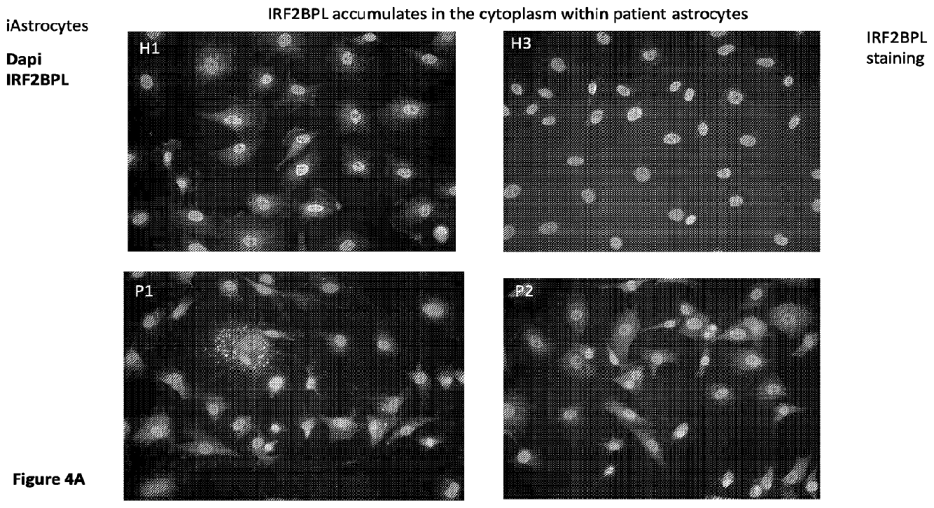

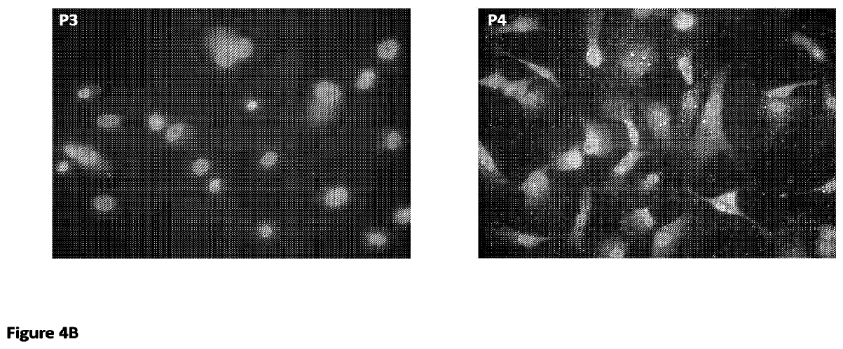

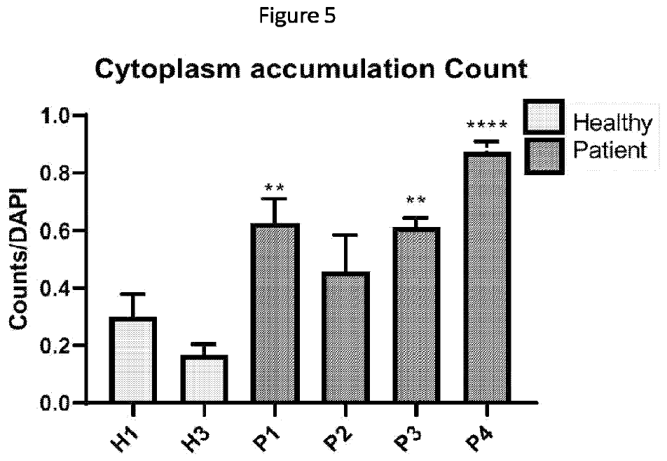

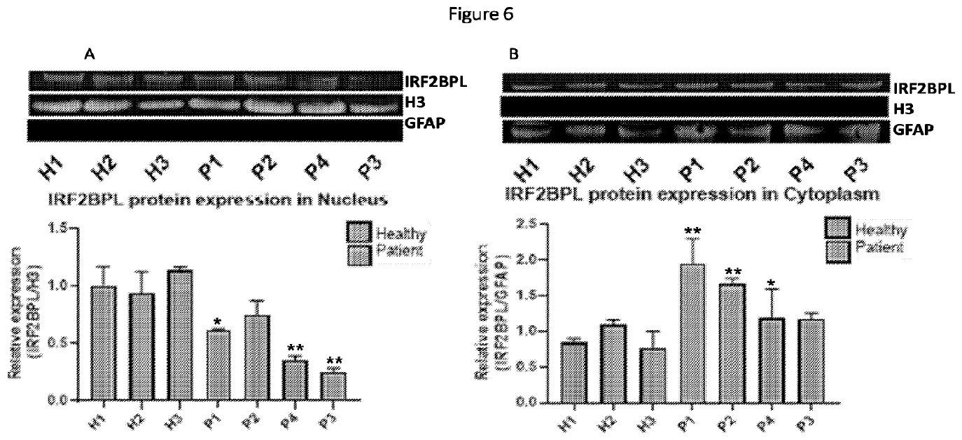

FIGS. 4A and B provide representative photos of the immunohistochemistry staining for IRF2BPL (red staining) and DAPI for the cell nucleus (blue), and FIG. 5 provides quantification of the aberrant cytoplasmic accumulation as observed from the immunofluorescence images. The normalized ratio is the number of cells with cytoplasm accumulation of IRF2BPL in astrocytes to the DAPI counts (n=3). Blinded-hand counting was carried out by two independent researchers. There was a clear difference in cytoplasmic IRF2BPL protein localized in patient cell lines. These photos and the graphs provided in FIG. 6 demonstrate that the IRF2BPL accumulated more in the cytoplasm of the iastrocytes derived from the NEDAMSS patients (P1, P2, P3 and P4) rather than localizing mostly to the nucleus of the iastrocytes like the healthy individuals (H1 and H3). NPER extraction kit was used to separate the two extracts and confirmed accumulation of the protein in the cytoplasm in patient astrocytes.

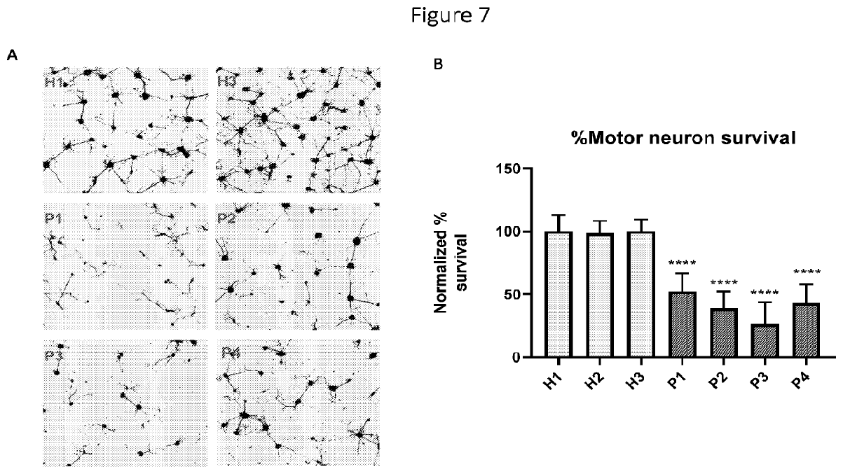

Coculture of iastrocytes with mouse stem cell derived GFP positive motor neurons (according to publication Meyer et al, PNAS 2014). Briefly, iastrocytes were plated in a 96 well plate to form a monolayer. The next day 10K, FACS sorted gfp positive mouse motor neurons are added to each well. Survival and morphology of neurons are monitored using the INCELL6000 automatic imager and analyzer software for 3 days. It was determined that astrocytes from NEDAMSS patients were toxic or less supportive to the motor neurons compared to iastrocytes from healthy controls. FIGS. 7A provides representative photos of the motor neurons in the coculture with iastrocytes from NEDAMSS patients and healthy individuals and 7B shows the percentage motor neuron survival. NEDAMSS astrocytes show significantly reduced motor neuron survival in the cocultures compared to healthy astrocytes on day 3. Of note, only the motor neurons are visible as they contain GFP (represented in black). In these photos, the motor neurons are visible due to GFP expression and the iastrocytes are not visible.

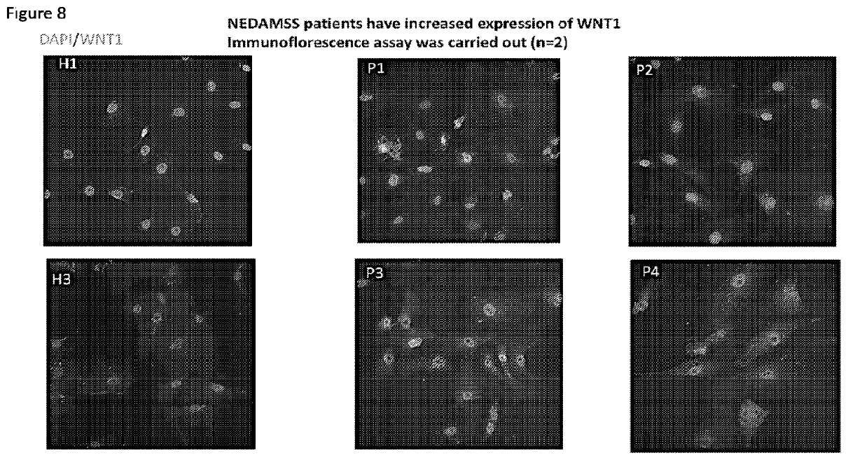

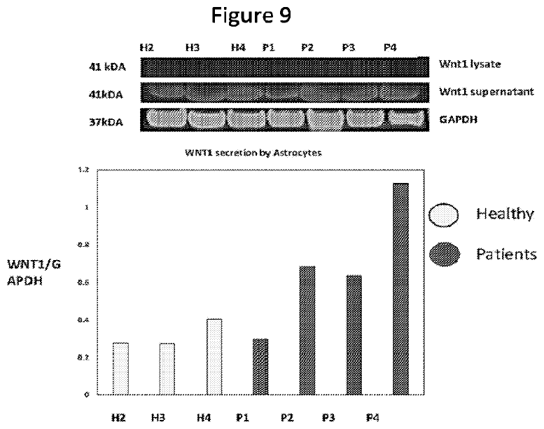

FIGS. 8 and 9 demonstrate that the NEDAMSS patients have increased secretion of WNT1 compared to healthy patients. Dysregulation of wnt signaling pathway could lead to neurodegeneration in NEDAMSS patients.

### Example 2—Neurons from NEDAMSS Patients have Reduced Survival

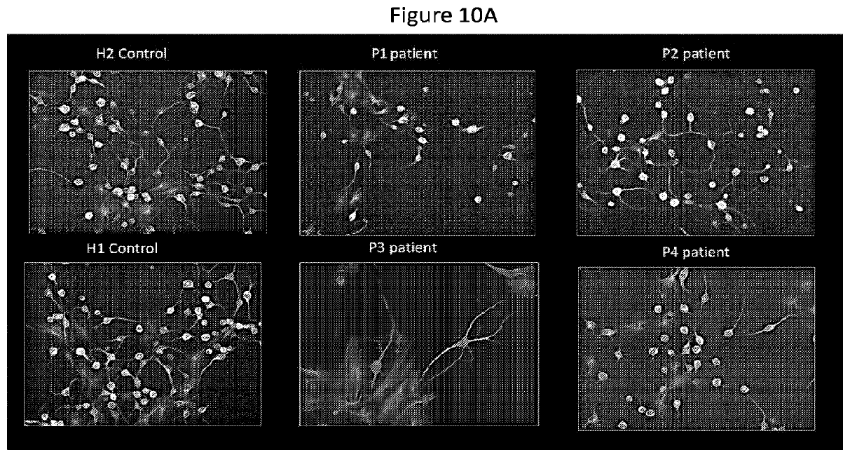

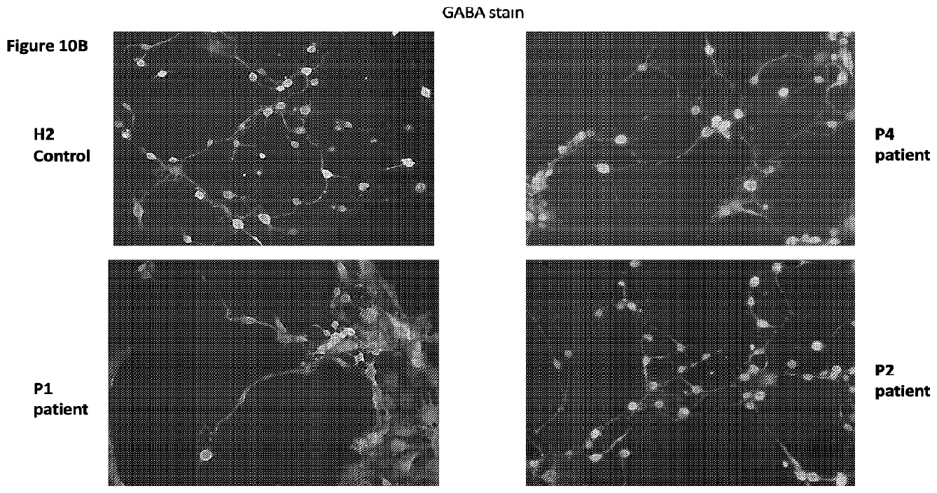

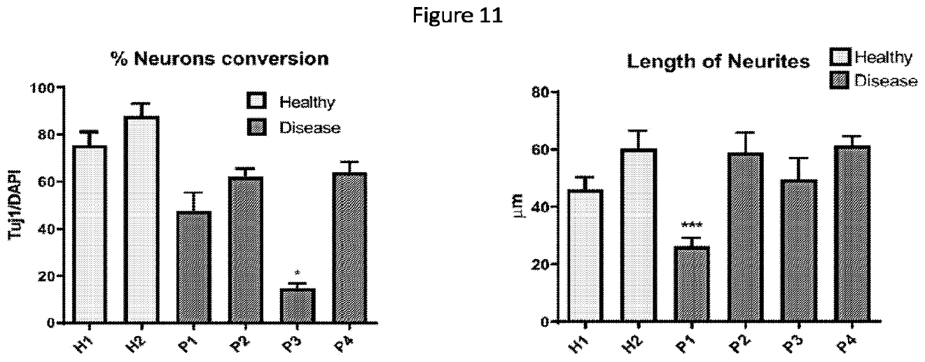

Fibroblasts isolated from healthy individuals and NEDAMSS patients were differentiated to neurons as described in Hu et al., Cell Stem Cell, 17 (2): 204-12, 2015, the disclosure of which is incorporated herein by reference in its entirety. The fibroblasts were incubated with 7 small molecules as described in Hu et al (supra) for 7 days. This method does not use transcriptional factor-expressing virus. FIGS. 10A and B provide representative photos showing that the neurons induced from fibroblasts from NEDAMSS patients had reduced survival or reduced differentiation capacity. FIG. 10A shows staining for the pan-neuron marker Tuj1 from NEDAMSS patients (P1, P2, P3 and P4) and healthy individuals (H1, and H2). FIG. 10B show staining for the neuronal subtype marker Gaba in neurons from NEDAMSS patients (P1, P2 and P3) and a healthy individual (H2). FIG. 11 provides quantification of the number of Tuj1+neurons (Tuj1) to DAPI and the length of the neurites from the NEDAMSS patients (P1, P2, P3 and P4) and healthy individuals (H1 and H2) on day 7 of differentiation.

### Example 3—Constructs Encoding IRF2BPL

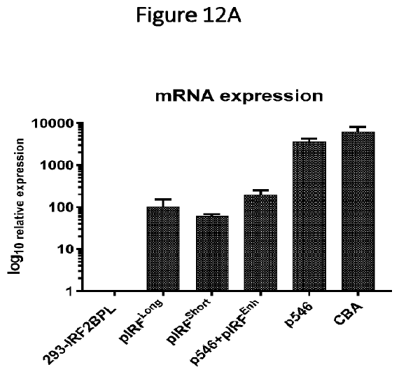

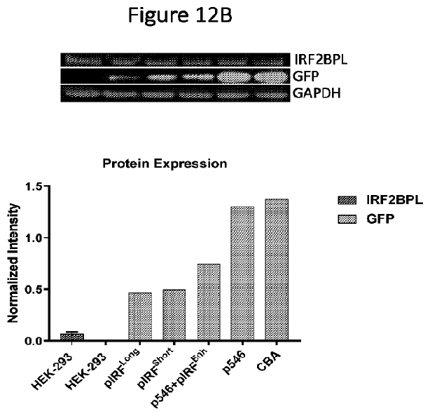

The human GFP cDNA clone was obtained from Origene, Rockville, MD. GFP cDNA alone was further subcloned into a self-complementary AAV9 genome under the control of one or more of either i) 1034 base pair IRF2BPL promoter (pIRF^{Long}), ii) 648 base pair fragment of the IRF promoter (pIRF^{Short}), iii) or a 296 base pair fragment of the IRF2BPL promoter (pIRF^{ENH}), iv) the p546 promoter or v) the hybrid chicken β-Actin promoter (CB). The plasmid construct also included an intron such as the simian virus 40 (SV40) chimeric intron, 907 base pairs of the 5′ untranslated region (UTR) of the full length IRF2BPL gene and a Bovine Growth Hormone (BGH) polyadenylation signal (BGH PolyA). The constructs were packaged into an AAV9 genome. These constructs were generated to investigate the regulatory control and strength of the promoters. Briefly, the plasmids were transiently transfected into human embryonic kidney cells using Calcium Phosphate. After incubation of several days, GFP was monitored using microscopes allowing visualization as well as the expression was quantified by qPCR and western blot. FIGS. 12A and 12B provides comparative IRF2BPL mRNA (FIG. 12A) and IRF2BPL (FIG. 12B) expression between wild-type IRF2BPL levels in HEK-293 and GFP levels as induced by the five different promoters.

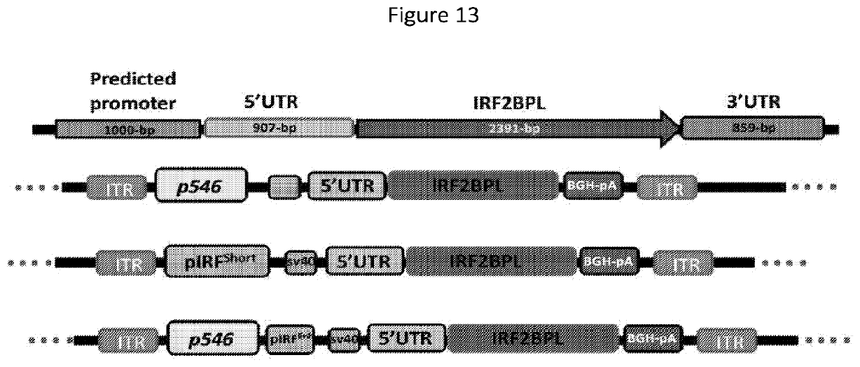

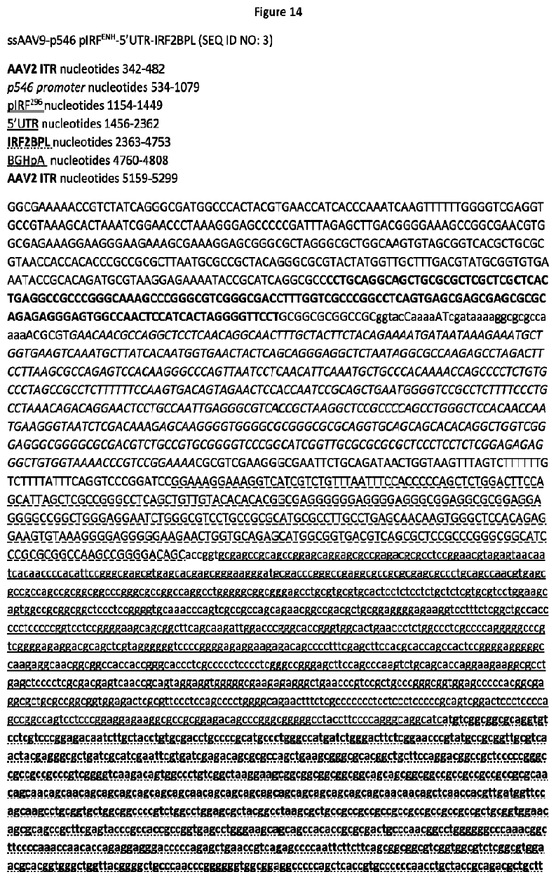

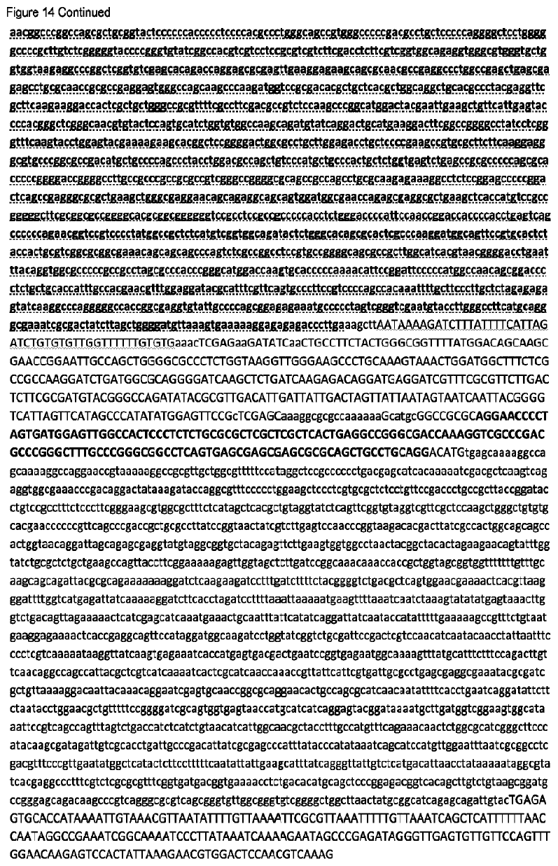

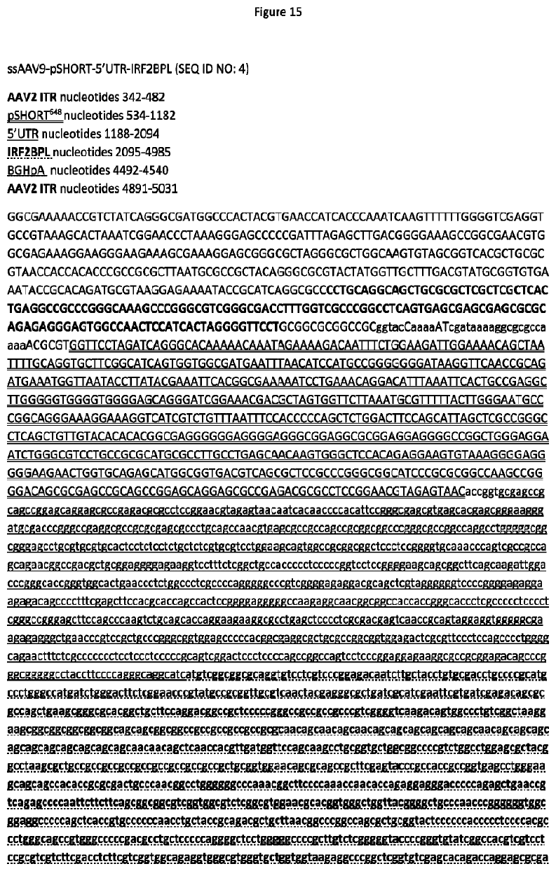

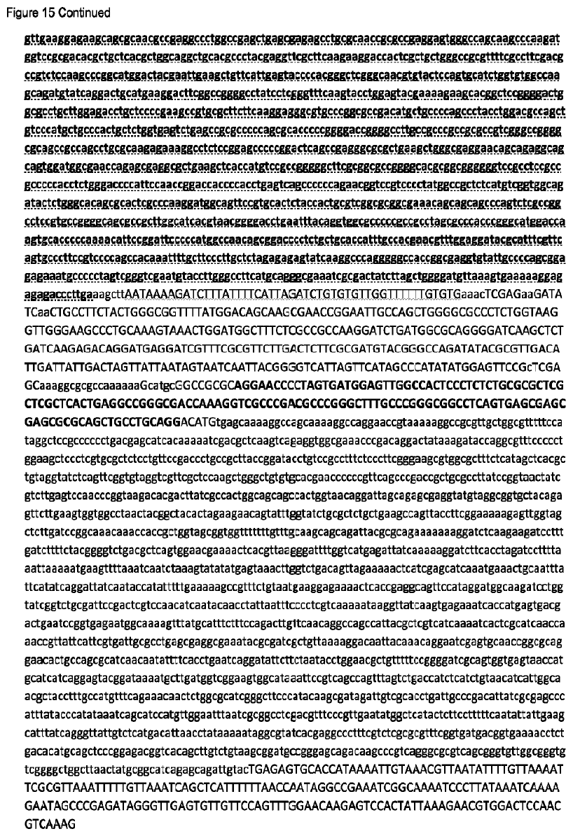

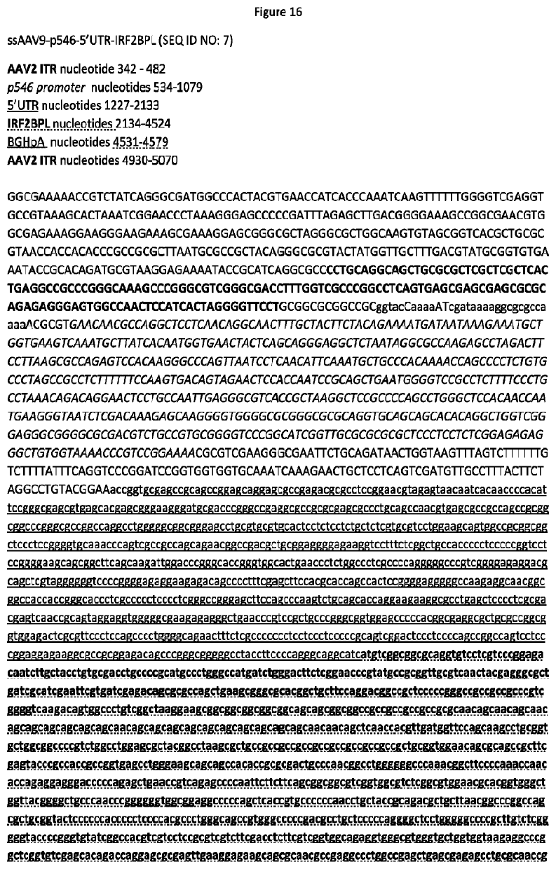

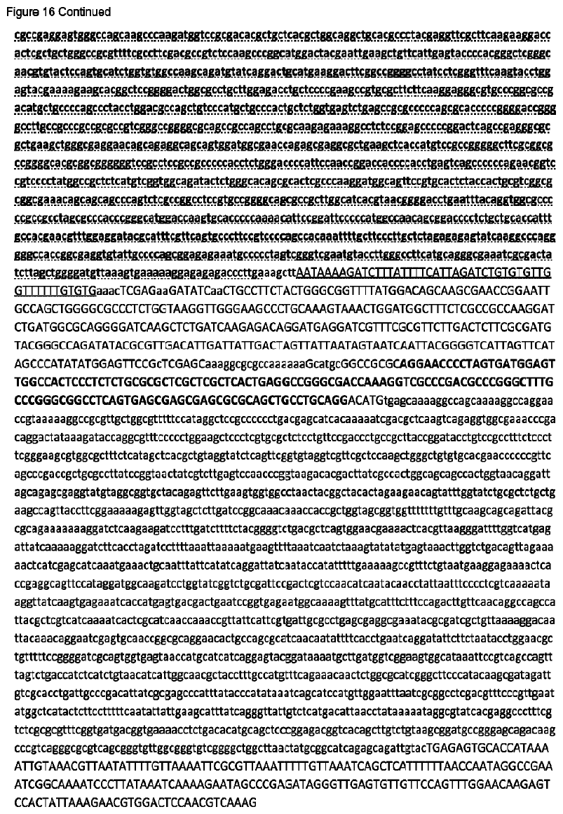

Exemplary polynucleotide constructs encoding IRF2BPL were generated as set forth in FIG. 13, the AAV9 vector design with the full-length transcript of IRF2BPL under the control of truncated variants of its endogenous promoter. The polynucleotide sequence of ssAAV9-p546+pIRF^{ENH}-5′UTR-IRF2BPL is set forth in SEQ ID NO: 3, in which the endogenous MECP2 promoter is followed by the truncated variant of the endogenous promoter pIRF²⁹⁶. The polynucleotide sequence of ssAAV9-pIRF^{SHORT}-5′UTR-IRF2BPL is set forth in SEQ ID NO: 4 in which the truncated variant of the endogenous promoter is the pIRF⁶⁴⁸. The polynucleotide sequence of ssAAV9-p546-5′UTR-IRF2BPL is set forth in SEQ ID NO: 7 in which the promoter is the endogenous MECP2 promoter. Annotated sequences showing the location of the construct elements are set out in FIGS. 14, 15 and 16 and summarized in the tables below.

ssAAV9-p546 pIRF^{ENH}-5′UTR-IRF2BPL (SEQ ID NO: 3)

| | |
|---|---|
| AAV2 ITR | nucleotides 342-482 |
| p546 promoter | nucleotides 534-1079 |
| pIRF²⁹⁶ | nucleotides 1154-1449 |
| 5′UTR | nucleotides 1456-2362 |
| IRF2BPL | nucleotides 2363-4753 |
| BGHpA | nucleotides 4760-4808 |
| AAV2 ITR | nucleotides 5159-5299 |

ssAAV9-pSHORT-5′UTR-IRF2BPL (SEQ ID NO: 4)

| | |
|---|---|
| AAV2 ITR | nucleotides 342-482 |
| pSHORT⁶⁴⁸ | nucleotides 534-1182 |
| 5′UTR | nucleotides 1188-2094 |
| IRF2BPL | nucleotides 2095-4985 |
| BGHpA | nucleotides 4492-4540 |
| AAV2 ITR | nucleotides 4891-5031 |

ssAAV9-p546-5′UTR-IRF2BPL (SEQ ID NO: 7)

| | |
|---|---|
| AAV2 ITR | nucleotides 342-482 |
| p546 promoter | nucleotides 534-1079 |
| 5′UTR | nucleotides 1227-2133 |
| IRF2BPL | nucleotides 2134-4524 |
| BGHpA | nucleotides 4531-4579 |
| AAV2 ITR | nucleotides 4930-5070 |

### REFERENCES

Marcogliese et al., IRF2BPL Is Associated with Neurological Phenotypes. The American Journal of Human Genetics, 2018, 103(3).
Meyer et al. Direct conversion of patient fibroblasts demonstrates non-cell autonomous toxicity of astrocytes to motor neurons in familial and sporadic ALS. PNAS, 2014, 2014, 111(2): 829-832
Hu et al. Direct Conversion of Normal and Alzheimer's Disease Human Fibroblasts into Neuronal Cells by Small Molecules. Cell Stem Cell, 2015, 17(2): 204-12.

1. A polynucleotide comprising
(a) one or more regulatory control elements; and
(b) an Interferon regulatory factor 2 binding protein like (IRF2BPL) cDNA sequence.

2. The polynucleotide of claim 1, wherein the regulatory control element is pIRF promoter, p546 promoter or CBA promoter, or fragments thereof.

3. (canceled)

4. (canceled)

5. A recombinant adeno-associated virus (rAAV) having a genome comprising a polynucleotide sequence of claim 1.

6. (canceled)

7. (canceled)

8. (canceled)

9. (canceled)

10. The rAAV of claim 5, wherein the rAAV is of the serotype AAV1, AAV2, AAV3, AAV4, AAV5, AAV6, AAV7, AAV8, AAV9, AAVRH10, AAVRH74, AAV11, AAV12, AAV13 or Anc80, AAV7m8 and their derivatives.

11. An rAAV particle comprising the rAAV of claim 5.

12. A method of treating an Interferon regulatory factor 2 binding protein like (IRF2BPL)-related disorder in a subject in need thereof comprising administering an rAAV of claim 5.

13. The method of claim 11, wherein the disorder comprises NEDAMSS (neurodevelopmental disorder with regression, abnormal movements, loss of speech, and seizures).

14. (canceled)

15. (canceled)

16. A method of treating an Interferon regulatory factor 2 binding protein like (IRF2BPL)-related disorder in a subject in need thereof comprising administering the rAAV particle of claim 11.

17. The method of claim 16, wherein the disorder comprises NEDAMSS (neurodevelopmental disorder with regression, abnormal movements, loss of speech, and seizures).
